(19) United States
(12) Patent Application Publication
(10) Pub. No.: US 2005/0214351 A1
(43) Pub. Date: Sep. 29, 2005

(54) Veterinary Bandage with Animal Repellant Incorporated Therein

(76) Inventor: Megan Chew, Santa Barbara, CA (US)

Correspondence Address: MICHAEL G PETIT, 123 W PADRE ST., SUITE F, P O BOX 91929, SANTA BARBARA, CA, 93190, US

(21) Appl. No.: 10/708,842

(22) Filed: Mar. 26, 2004

(51) Int. Cl. A61K 9/70

(52) U.S. Cl. 424/445

(57) ABSTRACT

The present invention relates to a veterinary wound dressing. The wound dressing comprises a standard veterinary bandage of the type normally employed to cover a wound. The bandage includes an outer surface and a wound-facing surface. In a first embodiment of the invention, a layer of fabric or a polymeric film containing an animal repellant substance is affixed to the outer surface of the bandage during the manufacturing process. Alternatively, in a second embodiment, a sheet of fabric or polymeric film having an adhesive lower surface and an opposing outer surface comprising an animal repellant substance is provided. The sheet or film may be applied to the outer surface of a prior art veterinary bandage. The animal repellant is preferably a non-toxic substance.

### BACKGROUND OF INVENTION

1. Field of the Invention

The present invention relates to a bandage for use in veterinary medicine, and, more particularly, to a bandage comprising animal repellant means operable for discouraging an animal from removing the bandage with its teeth.

2. Prior Art

An animal having undergone surgery generally has a wound or incision requiring a bandage. Because the bandage is a foreign object, the animal often tends to molest or attempt to remove the bandage material, resulting in the bandage becoming nonfunctional for the purpose of protecting the wound.

One way this problem has been addressed in the past is by the use of head cones to prevent the animal's access to the bandaged site with its mouth. However, headcones have many disadvantages. Oftentimes the cone must be worn for several days, thereby putting undue leverage and pressure on the animal's neck. Cones are impossible to use if the animal must be crated in order to limit activity. For domestic pets recovering at home, an animal having to endure the physical burden and visual limitations of a cone can easily damage furniture and harm themselves. The unnatural circumstance of having to endure a cone and an increased sense of vulnerability due to a reduced field of vision serves to stress many animals, thereby impeding their ability to recover from the surgery.

Another method of addressing this problem has involved the application of bitter tasting substances such as a gel sold under the tradename Yuk-2e applied directly to the animal's wound or bandage. Yuk-2e is a combination of denatonium and sucrose octaacetate and is extremely offensive to the animal, yet harmless. However, it requires re-application as frequently as every fifteen minutes. In both veterinary and home environments, this frequent need for attention puts an undue burden on the animal's caretaker and may serve to contaminate surfaces with which the animal comes into contact with. Clearly, there is a need for a bandaging device operable for protecting a surgical wound that is effective, convenient to use, includes a repellant that discourages the animal from removing the device with its mouth and minimizes additional trauma to the animal.

### SUMMARY OF INVENTION

It is an object of the present invention to provide a veterinary bandage operable for protecting a wound on an animal that includes means for discouraging the animal from removing the bandage with its teeth.

It is another object of the present invention to provide a veterinary bandage for protection of animal wounds that is safe, effective and convenient for the animal caretaker to use.

It is yet a further object of the present invention to provide a veterinary bandage for protection of animal wounds that will not traumatize the animal.

The above objectives are met by the provision of a veterinary dressing comprising a bandage and animal repellant substance wherein, in a first embodiment, the animal repellant substance is at least partially incorporated into the bandage at the time of manufacture and prior to packaging for dispensation. The veterinary dressing has a body facing surface and an outer surface. The outer surface is impregnated, coated or covered with the animal repellant at the time of manufacture. Preferably, the body facing surface and the outer surface of the veterinary dressing are separated by an intermediate layer that is substantially impermeable to the animal repellant. In a second embodiment, a film or sheet is provided having a body-facing adhesive surface that may be affixed to the outer surface of a bandage and an outer surface comprising an animal repellant. The features of the invention believed to be novel are set forth with particularity in the appended claims. However the invention itself, both as to organization and method of operation, together with further objects and advantages thereof may be best understood by reference to the following description taken in conjunction with the accompanying drawings.

### DETAILED DESCRIPTION

### BRIEF DESCRIPTION OF THE DRAWINGS

FIG. 1 is an illustration of a bandage wrapped around a dog's leg adjacent to a paw in accordance with the present invention.

FIG. 2 is a perspective view illustrating the bandage of FIG. 1 in an unwrapped configuration.

FIG. 3 is a cross-sectional view of one embodiment of the bandage of FIGS. 1 and 2 taken along section line 3-3.

FIG. 4 illustrates a possible method for incorporating an animal repellant within, or applying an animal repellant to, an outer surface of a bandage.

FIG. 5 illustrates a bandage treated in a manner consistent with the present invention, sterilized, packaged and ready for distribution.

### DESCRIPTION OF THE PREFERRED EMBODIMENTS

The definitions below serve to provide a clear and consistent understanding of the specification and claims, including the scope given to such terms.

The term “gel”, as used herein, means a viscous fluid having a viscosity in the range of about 100-10,000 cp.

The term “Yuk-2e”, as used herein, means a commercial animal repellant gel consisting of denatonium and sucrose octaacetate, C₂₈H₃₈O₁₉. Yuk-2e is commercially available through the internet from www.vetplanet.net.

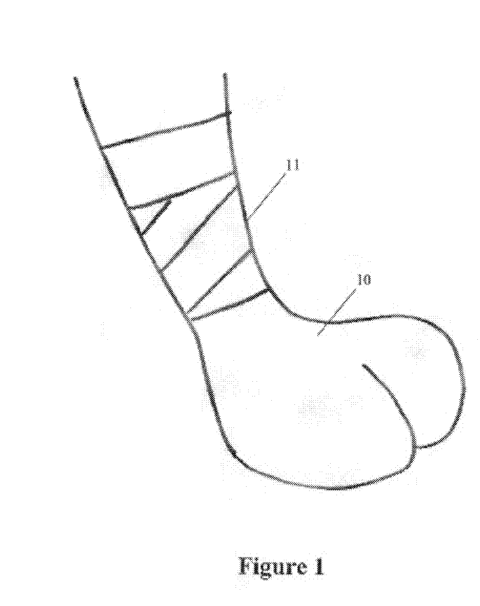

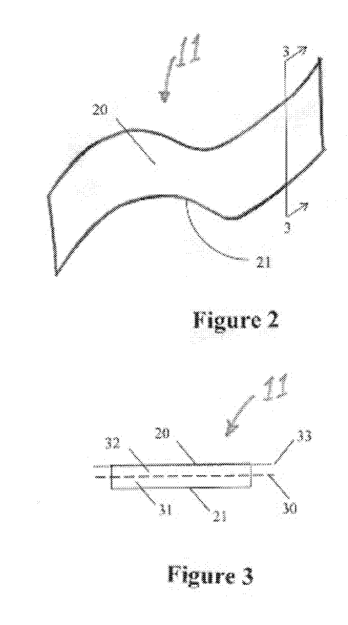

FIG. 1 shows a typical veterinary application for wound treatment wherein a bandage (11) is applied to a dog's leg adjacent to the paw (10) to cover and protect a wound (not shown). The bandage (11) is preferably an elongate strip of a flexible material having a sterile skin-facing surface (21) as illustrated in FIG. 2. FIG. 2 shows the bandaged unfurled. The underside (21) is the skin-facing surface of the bandage (11). The upper side (20) is the outward facing surface which is exposed to the animal's teeth. In one preferred embodiment, the entire bulk volume of the bandage material is impregnated with Yuk-2e or similar substance during the manufacturing process. This can be accomplished by soaking the bandage in a liquified form of the animal repellant substance prior to sterilization and packaging. Alternatively, the repellant of choice can be sprayed onto the upper side (i.e., the outward facing surface) (20). In the event that the bandage (11) comprises a fabric, the individual threads of the bandage can also be treated by soaking or spraying prior to being woven into a bandage configuration.

In a second preferred embodiment, the bandage is impregnated so that the Yuk-2e or similar substance is concentrated more towards the outward facing surface than the body facing surface during the manufacturing process. This can again be accomplished by spraying the animal repellant substance only on the outer surface (20) or by exposing only the outer side (20) of the bandage (11) to a liquified form of the repellant substance. In either case, the amount of repellant substance to which the outer side is exposed can control the degree to which it impregnates the bandage (11).

FIG. 3 is a cross-sectional view taken along section line 3-3 of FIG. 2 showing a preferred embodiment of the bandage of FIG. 2. An optional impermeable layer (30), preferably a polymeric film that retains its structural integrity when brought in contact with a repellant, is shown separating the bandage volume into two halves. The lower half (31) containing the skin-facing surface (21) is bounded by the body facing surface (21) and the impenetratable layer (30). The upper half (32) is bounded by the outward facing surface (20) and the impermeable layer (30). The impermeable layer (30) prevents the repellant substance in the upper half (32) of the bandage (11) from contacting the underlying skin. In a third preferred embodiment, only the upper half of the bandage is impreganted with Yuk-2e or similar substance during the manufacturing process. The upper half can be impregnated by any of the methods discussed above. An impermeable layer (30) may be used to prevent the repellant substance (33) such as Yuk-2e or similar substance from migrating into the lower half of the bandage.

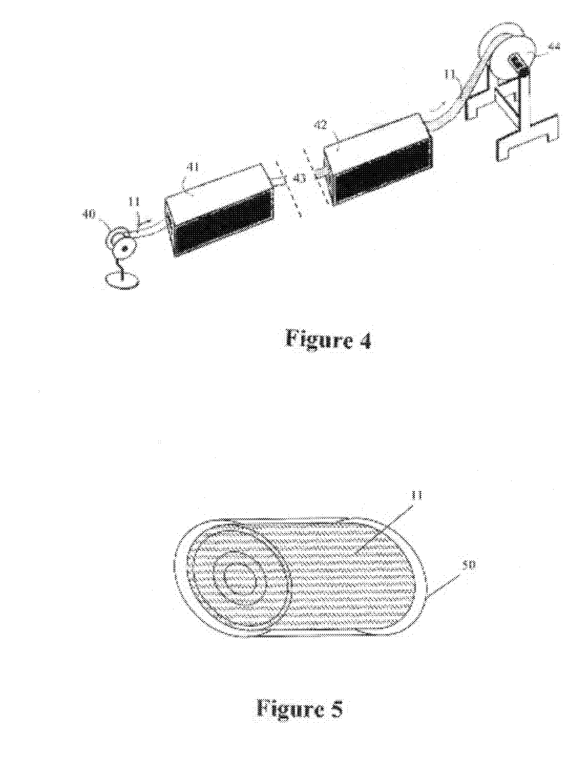

Turning now to FIG. 4, a simple process for incorporating the animal repellant (33) into the bandage material is illustrated in plan view. An untreated bandage (11) is fed from a bulk roll (40), and brought into and through the animal repellant application unit (41), wherein the bandage is soaked, sprayed, or otherwise treated with Yuk-2e or similar animal repellant substance. Upon leaving the application unit (41), the treated bandage material is subsequently fed into a sterilizing unit (42). Several optional processes (43) can be inserted between the application unit (41) and the sterilizing unit (42). For example, a second bandage can be laminated to the first with an optional impermeable layer inserted therebetween or a carrier/release layer can be applied to one or both bandage surfaces. After leaving the sterilizing unit, the bulk, finished product (11) is then rolled onto a take-up reel (44) for storage, secondary bulk cutting, or final shape cutting. The finished product, a sterilized bandage (11) appropriately packaged (50) for dispensation is illustrated in FIG. 5.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A veterinary dressing comprising a bandage and animal repellant substance wherein said animal repellant substance is at least partially incorporated into said bandage at the time of manufacture.

2. A veterinary dressing as in claim 1 wherein said bandage has a body facing surface and an outer surface, said outer surface being covered with said animal repellant at the time of manufacture.

3. A veterinary dressing as in claim 2 wherein said body facing surface and said outer surface are separated by a intermediate layer that is impermeable to the animal repellant substance.

4. A veterinary dressing in accordance with claim 2 wherein said body facing surface is covered with a carrier/release layer.

5. A veterinary dressing in accordance with claim 3 wherein said body facing surface is covered with a carrier/release layer.

6. A covering for a veterinary bandage comprising an elongate sheet having an animal repellant substance coated thereon.
